(19) United States
(12) Patent Application Publication
(10) Pub. No.: US 2011/0091935 A1
(43) Pub. Date: Apr. 21, 2011

(54) PRODUCTION OF RECOMBINANT PROTEINS IN SYNTHETIC MEDIUM

(75) Inventors: Patrick Erbacher, Benfeld (FR); Abdennaji Adib, Strasbourg (FR)

(73) Assignee: POLYPLUS TRANSFECTION SAS, Illkirch Cedex (FR)

(21) Appl. No.: 12/975,788

(22) Filed: Dec. 22, 2010

Related U.S. Application Data
(63) Continuation of application No. 11/884,477, filed on Sep. 24, 2007, now abandoned.
(60) Provisional application No. 60/654,003, filed on Feb. 18, 2005.

(51) Int. Cl. C12P 21/02

(52) U.S. Cl. 435/69.1

(57) ABSTRACT

The invention relates to a method for the production of recombinant proteins by eucaryotic cells in synthetic culture media, wherein said eucaryotic cells are transfected with a composition comprising a synthetic transfection reagent based on a polyhydroxylated polyalkyleneimine.

This application is a continuation of application Ser. No. 11/884,477 (pending), which was filed on Sep. 24, 2007 (published as US 2008-0160578-A1 on Jul. 3, 2008), which is a 371 U.S. national phase of International Application No. PCT/EP2006/002073, filed Feb. 16, 2006, which designated the U.S. and claims benefit of U.S. Provisional Application No, 60/654,003, filed Feb. 18, 2005, the entire contents of each of which are hereby incorporated by reference.

The invention relates to the production of recombinant proteins by eucaryotic cells in synthetic culture media.

Eucaryotic cell systems are currently used for the industrial production at large scale of recombinant proteins. HEK-293 and CHO cells, and the clones derivated therefrom are the most commonly used cell lines used and are cultivated either in suspension or under adherent form. To satisfy the regulation requirements, the production systems must be now devoid of any compound of animal origin. Serum-free culture media allowing HEK-293 and CHO culture have then been developed. However proteins were still added as proliferation factors. Totally synthetic culture media whose composition is totally defined, ie all the compounds are known, and none of them is of animal origin are advantageously used.

Specific transfection agents have been developed such as 293-Fectin® which specifically transfect HEK-293 cells in synthetic media such as FreeStyle (Invitrogen). However, said agents are only efficient for the culture medium they have been developed for.

A transfection agent having a broader spectrum has been largely used, ie PEI for polyethyleneimine.

The inventors have now found that derivatives of PEI were particularly efficient as transfection agents for the production of recombinant proteins by eucaryotic cells in any synthetic medium. Similar results have also been obtained with other polyalkyleneimine derivatives, particularly polypropyleneimine derivatives.

The invention thus aims to provide a new method for the production of recombinant proteins comprising the use of such derivatives.

The method according to the invention comprises the transfection of eucaryotic cells with a composition comprising a synthetic transfection reagent based on a polyhydroxylated polyalkyleneimine derivative.

The method according to the invention comprises a composition of transfection based on a highly hydrophilic polyalkyleneimine derivative.

Said polyalkyleneimine derivative is branched or linear. Said polyalkyleneimine group is linked to any hydroxylated derivative, for example via an amine, ester or amide linker.

Preferred polyalkyleneimines are polyethyleneimine (PEI in short) or polypropyleneimine (PPI in short).

Advantageously the polyhydroxylated structure contains carbohydrates such as monosaccharides, disaccharides or oligosaccharides.

The carbohydrate structure more particularly contains saccharides such as glucose, mannose, galactose or fucose residues.

The carbohydrate structure may also correspond to glucuronic acid esters or amides oligomers.

The polyhydroxylated structure may comprise hydroxylated alkyl motifs, such as ethoxyl groups.

Preferred transfection compositions further comprise one or several neutral polymer.

The MW of the polyalkyleneimines is preferably of about 1 to 1000 kDa.

The glycosidic chains grafted to the polyalkyleneimines comprise 1 to 20 osidic units.

In preferred transfection compositions, the hydroxylation extent of nitrogen residues of the polyalkyleneimines is of more than 0.1%. The hydroxylation extent may reach up to 80% or more with, for example, ethoxy or propoxy modified polymers.

The method of the invention is particularly useful for efficient and transient transfection of non adherent or adherent cells growing in synthetic media adapted for recombinant protein production.

Other characteristics and advantages of the invention will be given hereinafter and illustrated by the use of a transfection composition comprising as transfection agent: glycosylated PEI.

This agent will be designated hereinafter by “TA”.

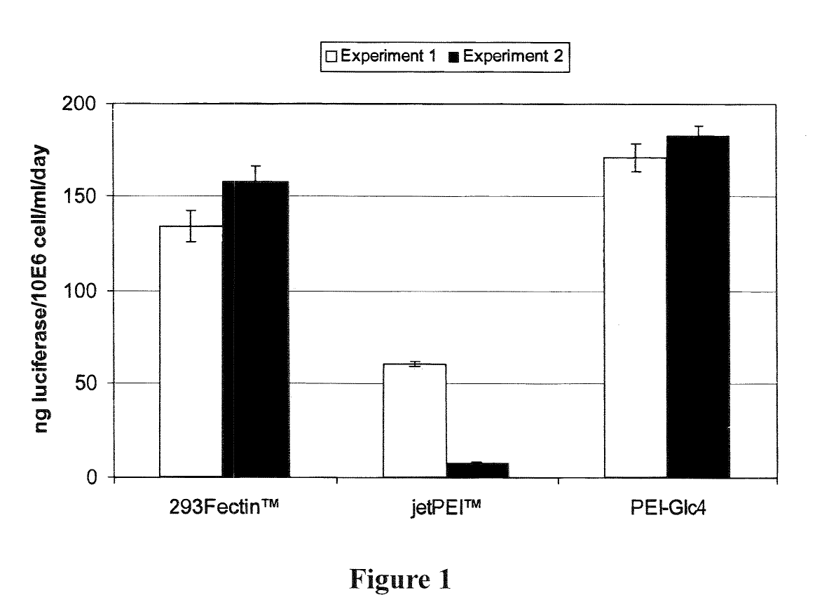

In said examples, it will be referred to FIGS. 1 to 5, which represent, respectively,

FIG. 1, the transfection of HEK-293 cells in suspension in synthetic medium,

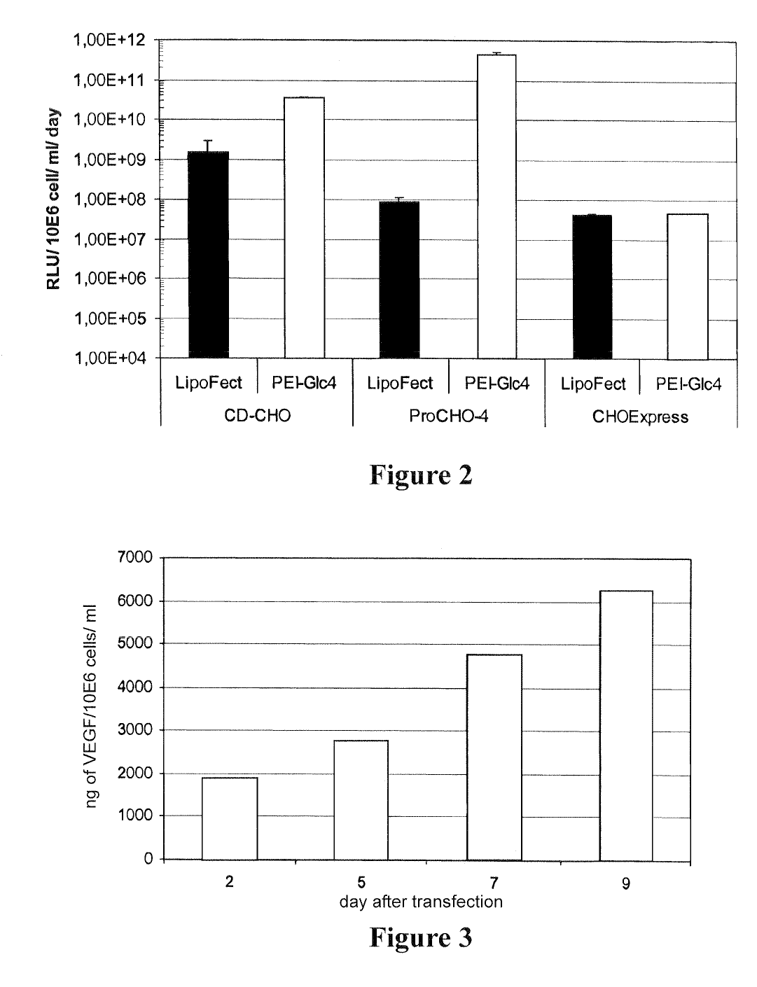

FIG. 2, the transfection of CHO cells in suspension in various synthetic media, and

FIGS. 3 to 5, VEGF production in CHO cells in suspension.

Synthesis of glycosylated linear PEI conjugates.

PEI was glycosylated by reductive amination procedure in the presence of sodium cyanoborohydride as previously described by Erbacher P. et al., 1999; 1:210-222 Transfection and physical properties of various saccharides, poly(ethylene glycol), and antibody derivatized polyethylenimines (PEI). S Gene Med. Linear tetraglucose (Glcα4-Glcα4-Glcα6-Glc, Sigma), or linear tetragalactose (Galα3-Galβ4-Galα6-Gal, Dextra Laboratories Ltd), or lactose (Glcα4-Gal, Sigma) (5 μmol) and sodium cyanoborohydride (25 mmol, Aldrich) were added to linear PEI (100 μmol of monomer, jetPEI™, Polyplus-Transfection SAS) in 0.9 ml of borate buffer 0.2 M, pH 8.2, and left for 96 h at room temperature under constant stirring. Low molecular weight compounds were removed on a Sephadex-G25 fine column (12.5×450 mm, Fluka) in 0.15 M NaCl. The grafting extent of different glycosyl structure per PEI molecule, expressed as a percentage of glycosyl motif per nitrogen, was determined by using the resorcinol/sulphuric acid micromethod and were 3-5%.

Transient Transfection of Suspension Cells

1. Suspension Culture Growth Conditions

Adaptation of Cells to Synthetic Medium

HEK-293 or CHO cells are grown in synthetic medium containing 4 mM glutamine, 1% of PLURONIC® F-68 (10% solution) with/without Penicillin/Streptomycin antibiotics. For cells adaptation in 50 ml of synthetic medium, cells are seeded at 500 000 cells per ml in 50 ml of synthetic medium in a 250 ml polycarbonate Erlenmeyer or shaker flask and incubated for 3-5 days at 37° C. in a humidified atmosphere with 8% CO₂ and under orbital constant shaking at 125 rpm.

Preparation of Cells for Transfection

Before transfection, cells are washed once with fresh synthetic medium, spun down and counted. Cells are seeded at 1 million per ml in FreeStyle™ (Invitrogen) medium and incubated at 37° C., 8% CO₂ under constant shaking in a total volume of 3 ml.

2. Preparation of TA/Plasmid DNA Complexes and Transfection Procedure

According to the synthetic medium used, the ratio between the transfection agent and the plasmid DNA may vary from 1 to 3 μl per μg of DNA. It is usually recommended to test two different amounts of plasmid DNA in order to determine optimal conditions. The amount of transfection reagent versus the amount of plasmid DNA for HEK 293 and CHO cells has been optimized with respect to some commercially available synthetic culture media. These optimized conditions are summarized in table 1. See table 2 for transfection with different DNA/transfection agent ratio.

TABLE 1

| | | Final volume | | Amount | | | |
|---|---|---|---|---|---|---|---|
| | | of | | of plasmid | | Amount of | |
| Cell line | Medium | cell culture | | DNA | | TA | |
| HEK 293 | * | per 1 | ml | 1 to 2 | μg | 1 to 2 | μl |
| | Freestyle ™ 293 | 50 | ml | 100 | μg | 100 | μl |
| CHO | * | per 1 | ml | 1 to 2 | μg | 1 to 2 | μl |
| | CD-CHO | 50 | ml | 100 | μg | 100 | μl |
| | ProCHO ™-4 | 50 | ml | 100 | μg | 300 | μl |
| | ProCHO ™-4 | 500 | ml | 1 | mg | 3 | ml |

*General starting conditions for optimization

2.1. Transfection Procedure for 1 ml of Cell Culture

Conditions are given for the transfection of 1×10⁶ suspension cells in 1 ml of synthetic medium. To perform transfection in larger volume of medium (1-10 ml), total cell number to seed as well as amounts of transfection reagent and plasmid DNA have to be changed proportionally.

Protocol for Transfection of 2 μg of DNA Per ml of Cell Culture.

Dilute 2 μg of plasmid DNA in 50 μl of synthetic medium (without PLURONIC® F-68 and antibiotics). Vortex for 10 seconds and incubate at room temperature for 15 minutes.

Dilute 2 μl of TA reagent in 50 μl of synthetic medium (without PLURONIC® F-68 and antibiotics). Vortex for 10 seconds and incubate at room temperature for 15 minutes.

Add the 50 μl TA solution to the 50 μl plasmid DNA solution rapidly and all at once

Vortex for 10 seconds.

Incubate for 30 minutes at room temperature, in order to allow complexes formation.

Add the 100 μl of TA/plasmid DNA complexes solution to the 1 ml of cell suspension (density: 1×10⁶ cells/nil) and incubate at 37° C. in a humidified atmosphere with 8% CO₂ and under orbital constant shaking at 125 rpm (adapt the cell culture vessel format to the final transfection volume. For 1-2 ml, use a 10 ml polycarbonate round bottom tube, for 3-10 ml use a 50 ml polycarbonate conic tube).

Maximal protein production is usually observed 48 h post-transfection.

2.2. Transfection Procedure for 50 ml of Cell Culture

it is recommend to test two different amounts of plasmid DNA; 50 and 100 μg (table 2) for optimisation. The following protocol is adapted for the transfection of 50×10⁶ suspension cells growing in 50 ml of synthetic medium (1×10⁶ cells/ml) with 100 μg of DNA and 100 μl of TA.

Dilute 100 μg of plasmid DNA in 2.5 ml of synthetic medium (without PLURONIC® F-68 and antibiotics). Vortex for 10 seconds and allow to stand at room temperature for 15 minutes.

Dilute 100 μl of TA in 2.4 ml of synthetic medium (without PLURONIC® F-68 and antibiotics). Vortex for 10 seconds and incubate at room temperature for 15 minutes.

Add the 2.5 ml TA solution to the 2.5 ml plasmid DNA solution rapidly and all at once.

Mix immediately with a Vortex for 10 seconds.

Incubate for 30 minutes at room temperature, in order to allow complexes formation.

Add the 5 ml of TA/plasmid DNA complexes solution to the 50 ml of cell suspension (density: 1×10⁶ cells/ml) in a 250 ml shaker flask (polycarbonate Erlenmeyer flask) and incubate at 37° C. in a humidified atmosphere with 8% CO₂ and under orbital constant shaking at 125 rpm.

Maximal protein production is usually found at 48 h post-transfection.

To perform transfection in a larger volume of medium, total cell numbers for seeding as well as amounts of transfection reagent and plasmid DNA have to be changed proportionally.

TABLE 2

| | | | | Volume of | | |
|---|---|---|---|---|---|---|
| | | | | medium | | |
| Amount | Ratio | Volume | | for TA | Total | Final |
| of | of DNA/ | of TA | | and DNA | volume of | volume of |
| DNA | TA | reagent (μl) | | dilution | complexes | cell culture |
| 50 μg | 1/1 | 50 | | 2.5 ml | 5 ml | 50 ml |
| | 1/3 | 150 | | 2.5 ml | 5 ml | 50 ml |
| 100 μg | 1/1 | 100 | | 2.5 ml | 5 ml | 50 ml |
| | 1/3 | 300 | | 2.5 ml | 5 ml | 50 ml |
| 1 mg | 1/1 | 1 | ml | 15 ml | 30 ml | 500 ml |
| | 1/3 | 3 | ml | 15 ml | 30 ml | 500 ml |

Transient Transfection of Adherent Cells

1. Cell Seeding

For optimal transfection conditions with TA, cells should be 50-60% confluent. Typically, for transfection in 24-well plates, 50 000 cells are seeded per well, 24 hours before transfection. For other culture formats, refer to table 3 for the recommended number of cells to seed the day before transfection.

Table 3 gives complex preparation for different cell culture formats.

TABLE 3

| | | | Volume of | | | | | |
|---|---|---|---|---|---|---|---|---|
| | | | medium | | Volume | Total | | |
| | | Amount of | for TA | | of TA | volume of | | Total |
| | Nb of cells | plasmid DNA | and DNA | | reagent (μl) | complexes | | volume of |
| Culture vessel | to seed | (μg) | dilution | | ratio 1/1) | per well | | medium |
| 24-well | 50 000 | 2 | 50 | μl | 2 | 100 | μl | 1 ml |
| 12-well | 100 000 | 4 | 50 | μl | 4 | 100 | μl | 2 ml |
| 6-well/35 mm | 200 000 | 8 | 100 | μl | 8 | 200 | μl | 4 ml |
| 10 cm | 1 000 000 | 25 to 40 | 500 | μl | 25-40 | 1000 | μl | 20 ml |
| 14 cm/153 cm² | 4 × 10⁶ − 10⁷ | 50 to 100 | 2 | ml | 50 to 100 | 4 | ml | 40 ml |
| Multitray | 10⁷ − 4 × 10⁷ | 300 to 500 | 20 | ml | 300 to 500 | 40 | ml | 200 ml* |
| 630 cm² | | | | | | | | |

*add synthetic medium up to 400 ml, 2 to 4 h after transfection

2. Preparation of TA/DNA Complexes and Transfection Procedure

2.1. Transfection Procedure for 1 ml of Cell Culture

The following protocol is given for transfection in 24-well tissue plates, refer to table 3 for transfection in other culture formats. Conditions are given for the transfection of 50 000 cells in 1 ml of synthetic medium. During optimisation we recommend testing two different amounts of plasmid DNA; 2 and 1 μg. The following protocol is prepared for the transfection of 2 μg of DNA.

Dilute 2 μg of plasmid DNA in 50 μl of synthetic medium (without PLURONIC® F-68 and antibiotics). Vortex for 10 seconds and allow to stand at room temperature for 15 minutes.

Dilute 2 μl of TA in 50 μl of synthetic medium (without PLURONIC® F-68 and antibiotics). Vortex for 10 seconds and incubate at room temperature for 15 minutes.

Add the 500 TA solution to the 50 μl plasmid DNA solution rapidly and all at once.

Mix immediately with a Vortex for 10 seconds.

Incubate for 30 minutes at room temperature, in order to allow complexes formation.

Add the 100 μl of TA/plasmid DNA complexes solution drop-wise in each well and homogenize the mixture by gently swirling the plate.

Incubate at 37° C. in a humidified atmosphere with 5 or 8% CO₂.

Transfection experiments are usually stopped after 24 to 48 hours and protein expression is assessed.

Transfection of Non-Adherent HEK-293 Cells in Suspension in Synthetic Medium.

Cells are seeded at 1 million per ml in FreeStyle™ (Invitrogen) medium and incubated at 37° C., 8% CO₂ under constant shaking in a total volume of 3 ml. Cells were transfected with 6 μg of plasmid DNA (pCMVLuc) and 6 μl of PEI-Glc4 (linear tetraglucosyl PEI derivative). 293Fectin™ (from Invitrogen) and jetPEI™ (linear polyethylenimine, from Polyplus-Transfection) were used according to the manufacturer's recommendations. Luciferase protein content (ng of luciferase) is quantified 24 hr post-transfection. Each transfection was done in triplicate.

The results are given on FIG. 1.

The luciferase level obtained with the PEI-Glc4 was hardly higher than the level obtained with the unmodified linear polyethylenimine (jetPEI™) and compared also favourably with the level obtained with the 293-Fectin™, specially designed for this protein production application.

Transfection of Non-Adherent CHO Cells in Suspension in Various Synthetic Media.

Cells were seeded at 1 million per ml in either CD-CHO (Invitrogen), ProCHO™-4 (Cambrex) or CHOExpress (PAA Laboratories) medium and incubated at 37° C., 8% CO₂ under constant shaking in a total volume of 3 ml. Cells were transfected with 6 μg of pCMVluc DNA and 18 μl of PEI-Glc4 or 30 p. 1 of LipoFectAmine-2000 (LipoFect, Invitrogen). Luciferase expression (RLU) was assayed 24 h after transfection. Each experiment was done in triplicate.

The results are given on FIG. 2 and show that PEI-Glc4 is more efficient in transfection compared with LipofectAmine-2000.

Cells were seeded at 1 million per ml in ProCHO™-4 (Cambrex) medium in a total volume of 50 ml and incubated at 37° C., 8% CO₂ under constant shaking. Cells were transfected with 2 μg/ml of pCMVhVEGF₁₆₅ DNA (coding for human vascular growth factor) and 3 μl of PEI-Glc4 per μg of DNA. Amount of VEGF protein produced was quantified by ELISA at different time after transfection. Each quantification is done in triplicate.

The results are given on FIG. 3. The quantification of VEGF secreted shows a sustained production over many days, confirming the potency of PEI-Glc4 application for a protein production of therapeutic interest.

In other experiments,

Cells were seeded at 1 million per ml in ProCHO™-4 (Cambrex) medium in a different total volume of 3, or 50 ml, or 500 ml and incubated at 37° C., 8% CO₂ under constant shaking. Cells were transfected with 2 μg/ml of pCMVhVEGF₁₆₅ DNA (coding for human vascular growth factor) and 3 p. 1 of PEI-Glc4 per μg of DNA. Amount of VEGF protein produced was quantified by ELISA at different time after transfection. Each quantification is done in triplicate.

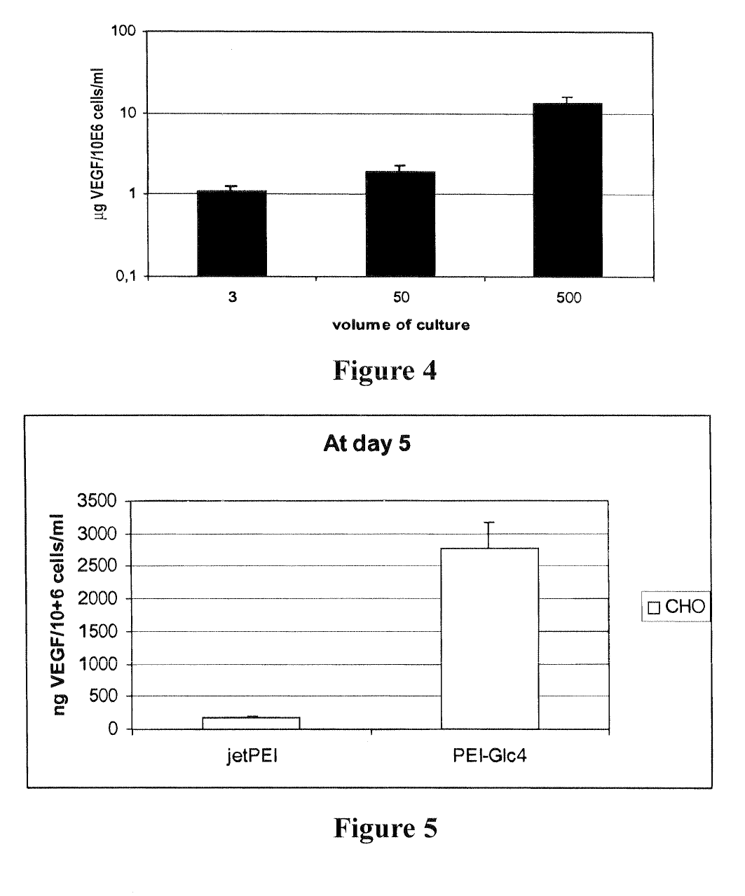

The results are given on FIG. 4 and show that PEI-Glc4 is well-adapted for the scale-up of protein production in different volume of bioreactors without affecting its transfection property.

In still other experiments,

Cells were seeded at 1 million per ml in ProCHO™-4 (Cambrex) medium in a total volume of 50 ml and incubated at 37° C., 8% CO₂ under constant shaking. Cells were transfected with 2 μg/ml of pCMVhVEGF₁₆₅ DNA (coding for human vascular growth factor) and 3 μl of PEI-Glc4 or jetPEI™ per μg of DNA. Amount of VEGF protein produced was quantified by ELISA at day 5 after transfection. Each quantification is done in triplicate.

The results are given in FIG. 5. A comparison is presented between non-adherent CHO cells in synthetic ProCHO™-4 medium transfected with PEI-Glc4 derivative and linear PEI (jetPEI™). The results shows a very low level of VEGF production following transfection with unmodified linear PEI compared with the level obtained with PEI-Glc4.

Table 4 gives the effect of the cell density on the VEGF production 2 days after transfection of non-adherent CHO cells in 3 ml of synthetic media. The transfections were performed with 2 μg/ml of pCMVLuc plasmi and with 3 μl/μg of DNA for each transfection reagent. The VEGF amount was determined by ELISA. Each experiment is done in triplicate.

TABLE 4

| Transfection | Synthetic | | DNA | ng of |
|---|---|---|---|---|
| reagent | medium | Cell density | amount | VEGF/10 * 6 cells/ml |
| PEI-Glc4 | CDCHO | 10 * 6/ml | 2 μg/ml | 72 |
| | | 2.10 * 6/ml | 2 μg/ml | 123 |
| | | 3.10 * 6/ml | 2 μg/ml | 19 |
| PEI-Glc4 | ProCHO4 | 10 * 6/ml | 2 μg/ml | 1082 |
| | | 2.10 * 6/ml | 2 μg/ml | 276 |
| | | 3.10 * 6/ml | 2 μg/ml | 243 |

Table 4 presents examples of optimization of transfection conditions of non-adherent CHO cells when TA is used. The results show a transfection conditions adapted to high density cell culture (>500,000 cells/ml) normally applied for the recombinant protein production processes.

Table 5 gives the effect of glycosyl motif grafted to linear PEI on the transfection efficiency of non adherent CHO cells in 3 ml of ProCHO-4 medium. The transfections were performed with 2 μg/ml of pCMVLuc plasmi and with 3 μl/μg of DNA for each transfection reagent. The luciferase activity was determined 48 h post-transfection. Each experiment is done in triplicate.

TABLE 5

| | | RLU/mg of | |
|---|---|---|---|
| Transfection reagent | Glycosyl motif | protein | SD |
| Linear PEI | none | 3.37E+4 | 2.15E+3 |
| (jetPEI ™) | | | |
| PEI-Glc4 | Glcα4-Glcα4-Glcα6- | 1.40E+7 | 5.15E+5 |
| | Glc | | |
| PEI-Gal4 | Galα3-Galβ4-Galα6- | 2.45E+7 | 7.57E+5 |
| | Gal | | |
| PEI-Lact | Glcα4-Gal | 1.86E+8 | 5.26E+6 |
| BPEI-Etox 80% | none | 1.60E+6 | 3.25E+4 |

The table 5 presents the activity level of luciferase protein produced in non-adherent CHO cells in ProCHO™-4 medium after transient transfection of pCMVLuc plasmid using various glycosylated derivatives of linear PEI. The structure of glycosyl motifs grafted on linear PEI can be different without affecting greatly the overall level of protein production. The osidic nature identifying the glycosyl motif, such as glucosyl or galactosyl residue, has no major effect on the recombinant protein level produced by transfection. Indeed, all glycosyl derivatives of linear PEI are more efficient than the unmodified linear PEI.

We claim:

1. A method for the production of recombinant proteins by eucaryotic cells in synthetic culture media, wherein said eucaryotic cells are transfected with a composition comprising a synthetic transfection reagent based on a polyhydroxylated polyalkyleneimine.

2. The method of claim 1, wherein said polyalkyleneimine derivative is branched or linear.

3. The method of claim 1, wherein said polyalkyleneimine group is linked to any hydroxylated derivative, for example via an amine, ester or amide linker.

4. The method of claim 1, wherein said polyalkyleneimine is polyethyleneimine (PH) or polypropyleneimine (PPI).

5. The method of claim 1, wherein the polyhydroxylated structure contains carbohydrates such as monosaccharides, disaccharides or oligosaccharides.

6. The method of claim 1 wherein the carbohydrate structure contains saccharides such as glucose, mannose, galactose or fucose residues.

7. The method of claim 1, wherein the carbohydrate structure corresponds to glucuronic acid esters or amides oligomers.

8. The method of claim 1, wherein the polyhydroxylated structure comprises hydroxylated alkyl motifs, such as ethoxyl groups.

9. The method of claim 1, wherein the transfection compositions further comprise one or several neutral polymer.

10. The method of claim 1, wherein the MW of the polyalkyleneimines is of 1 to 1000 kDa.

11. The method of claim 1, wherein the polyhydroxylation extent of nitrogene residues of the polyalkyleneimines is more than 0.1%.
